(19) United States
(12) Patent Application Publication
(10) Pub. No.: US 2007/0293475 A1
(43) Pub. Date: Dec. 20, 2007

(54) ARYL AND HETEROARYL TETRAHYDROBENZAZEPINE DERIVATIVES AND THEIR USE FOR TREATING GLAUCOMA

(75) Inventors: Suchismita Mohapatra, Arlington, TX (US); Mark R. Hellberg, Arlington, TX (US); Zixia Feng, Arlington, TX (US)

Correspondence Address: ALCON, IP LEGAL, TB4-8, 6201 SOUTH FREEWAY, FORT WORTH, TX, 76134, US

(73) Assignee: ALCON MANUFACTURING LTD., Fort Worth, TX (US)

(21) Appl. No.: 11/761,493

(22) Filed: Jun. 12, 2007

Related U.S. Application Data
(60) Provisional application No. 60/814,971, filed on Jun. 20, 2006.

(51) Int. Cl. A61K 31/55

(52) U.S. Cl. 514/217.01

(57) ABSTRACT

Aryl tetrahydrobenzazepine derivatives with minimal 5-HT_{2B} activity relative to 5-HT_{2A} and 5-HT_{2C} activity that are useful for treating glaucoma are disclosed.

This application claims priority to U.S. Provisional Application, U.S. Ser. No. 60/814,971 filed Jun. 20, 2006.

The present invention is directed to compounds useful for treating ophthalmic diseases. In particular, the present invention is directed toward aryl and heteroaryl tetrahydrobenzazepine derivatives and their use for lowering and controlling intraocular pressure (IOP) and treating glaucoma.

### BACKGROUND OF THE INVENTION

The disease state referred to as glaucoma is characterized by a permanent loss of visual function due to irreversible damage to the optic nerve. The several morphologically or functionally distinct types of glaucoma are typically characterized by elevated IOP, which is considered to be causally related to the pathological course of the disease. Ocular hypertension is a condition wherein intraocular pressure is elevated, but no apparent loss of visual function has occurred; such patients are considered to be a high risk for the eventual development of the visual loss associated with glaucoma. Some patients with glaucomatous field loss have relatively low intraocular pressure. These so called normotension or low tension glaucoma patients can also benefit from agents that lower and control IOP. If glaucoma or ocular hypertension is detected early and treated promptly with medications that effectively reduce elevated intraocular pressure, loss of visual function or its progressive deterioration can generally be ameliorated. Drug therapies that have proven to be effective for the reduction of intraocular pressure include both agents that decrease aqueous humor production and agents that increase the outflow facility. Such therapies are in general administered by one of two possible routes, topically (direct application to the eye) or orally.

There are some individuals who do not respond well when treated with certain existing glaucoma therapies. There is, therefore, a need for other topical therapeutic agents that control IOP.

It has been found that serotonergic compounds which possess agonist activity at 5-HT₂ receptors effectively lower and control normal and elevated IOP and are useful for treating glaucoma, see U.S. Pat. No. 6,664,286. Compounds that act as agonists at 5-HT₂ receptors are well known and have shown a variety of utilities, primarily for disorders or conditions associated with the central nervous system (CNS). U.S. Pat. No. 5,494,928 discloses certain 2-(indol-1-yl)-ethylamine analogs that are 5-HT_{2C} agonists for the treatment of obsessive compulsive disorder and other CNS derived personality disorders. U.S. Pat. No. 5,571,833 discloses tryptamine analogs that are 5-HT₂ agonists for the treatment of portal hypertension and migraine. U.S. Pat. No. 5,874,477 discloses a method for treating malaria using 5-HT_{2A/2C} agonists. U.S. Pat. No. 5,902,815 discloses the use of 5-HT_{2A} agonists to prevent adverse effects of NMDA receptor hypo-function. WO98/31354A2 discloses 5-HT_{2B} agonists for the treatment of depression and other CNS Is conditions. Agonist response at the 5-HT_{2A} receptor is reported to be the primary activity responsible for hallucinogenic activity, with some lesser involvement of the 5-HT_{2C} receptor possible [Psychopharmacology, Vol. 121:357, 1995].

WO 2005042491 discloses the following compounds as selective 5-HT_{2C} agonists useful for the treatment of obesity and related disorders:

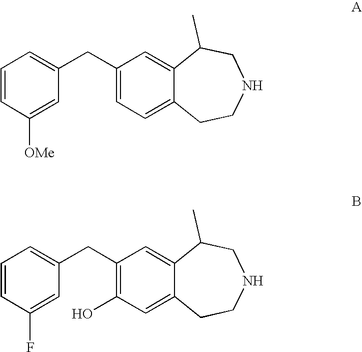

Additionally, WO 2002074746 and WO 199300094 disclose series of nor-methyl tetrahydrobenzazepine analogs useful in the treatment of disorders characterized by excessive vasodilation. None of these references describes the use of the substituted aryl tetrahydrobenzazepines of the present invention for treating glaucoma.

### SUMMARY OF THE INVENTION

The present invention is directed toward certain aryl and heteroaryl tetrahydrobenzazepine derivatives that can be used to lower and control IOP and treat glaucoma in warm blooded animals, including man. The compounds are preferably formulated in pharmaceutical compositions suitable for topical delivery to the eye.

Among other factors, the present invention is based on the finding that compounds that function as 5-HT_{2A/2C} agonists with low or no 5-HT_{2B} agonist potency (reported to be responsible for cardiovascular side effects) can be designed.

### DESCRIPTION OF THE PREFERRED EMBODIMENTS

Compounds that are useful for lowering and controlling normal or elevated IOP and treating glaucoma according to the present invention are represented by the following formula:

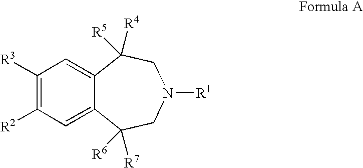

wherein
R¹═H or C₁₋₄ alkyl;
R²═H, OH, or OR where R═C₁₋₄ alkyl;
R³═—X—Ar, —OR⁸, —(CH₂)ₙOR⁸, or —(CH₂)_{n′}—O—(CH₂)ₘOR⁸;
R⁴, R⁵═H or C₁₋₂ alkyl;
R⁶, R⁷═H or C₁₋₂ alkyl;
when R⁴ or R⁵═C₁₋₂ alkyl, then R⁶═R⁷═H;
when R⁶ or R⁷═C₁₋₂ alkyl, then R⁴═R⁵═H;
R⁸, R⁹, R¹⁰═H or C₁₋₄ alkyl;
n=1-4;
n′=1-4;
m=1-4;
X═O, —C(R⁹)(R¹⁰)—, —OC(R⁹)(R¹⁰)—, or —C(R⁹)(R¹⁰)O—;
Ar=phenyl, optionally mono- or di-substituted with F, Cl, Br, I, C₁₋₄ alkyl, OH, or OR⁸; or 2-, 3-, 4-pyridyl, optionally mono- or di-substituted with F, Cl, Br, I, C₁₋₄ alkyl, OH, or OR⁸;
and pharmaceutically acceptable salts thereof.

Novel compounds of formula A are those wherein:
R¹═H;
R²═H, OH, or OR where R═C₁₋₄ alkyl;
R³═—X—Ar, —OR⁸, —(CH₂)ₙOR⁸, or —(CH₂)_{n′}—O—(CH₂)ₘOR⁸;
R⁴, R⁵═H;
R⁶, R═H;
R⁸═C₁₋₄ alkyl;
R⁹, R¹⁰═H or C₁₋₄ alkyl;
n=1-4;
n′=1-4;
m=2-4;
X═O, —C(R⁹)(R¹⁰)—, —OC(R⁹)(R¹⁰)—, or —C(R⁹)(R¹⁰)O—;
Ar=phenyl, optionally mono- or di-substituted with F, Cl, Br, C₁₂ alkyl, OH, or OR⁸; or 2-, 3-, 4-pyridyl, optionally mono- or di-substituted with F, Cl, Br, I, C₁₋₄ alkyl, OH, or OR⁸;
and pharmaceutically acceptable salts thereof.

Pharmaceutically acceptable addition salts include pharmaceutically acceptable acid addition salts prepared from acids including but not limited to acetic acid, benzenesulfonic acid, citric acid, fumaric acid, hydrobomic acid, hydrochloric acid, maleic acid, tartaric acid, phosphoric acid, sulfuric acid and the like. The acid addition salts may be obtained as the direct product of compound synthesis. Alternatively, the free base may be dissolved in a suitable solvent containing the appropriate acid, and the salt isolated by evaporation of the solvent or otherwise separating the salt and solvent. The compound of this invention may form solvates with standard low molecular weight solvents using methods know to those skilled in the art.

It is recognized that compounds of Formula A can contain one or more chiral centers. This invention contemplates all enantiomers, diastereomers, and mixtures thereof.

In the above definitions, the total number of carbon atoms in a substituent group is indicated by the Cᵢ₋ⱼ prefix where the numbers i and j define the number of carbon atoms; this definition includes straight chain, branched chain, and cyclic alkyl or (cyclic alkyl)alkyl groups.

Preferred compounds of Formula A are those in which:
R₁═H;
R₂═OH, or OR, where R═C₁₋₄ alkyl;
R₃═—X—Ar, —OR⁸, —(CH₂)ₙOR⁸, or —(CH₂)ₙ—O—(CH₂)ₘOR⁸;
R⁴, R⁵═H or C₁ alkyl;
R⁶, R⁷═H or C₁ alkyl;
when R⁴ or R⁵═C₁ alkyl, then R⁶═R⁷═H;
when R⁶ or R⁷═C, alkyl, then R⁴═R⁵═H;
R⁸═C₁₋₄ alkyl;
R⁹, R¹⁰═H or C₁₋₄ alkyl; and
Ar=phenyl, optionally mono- or di-substituted with F, Cl, Br, C₁₋₂ alkyl, OH, or OR⁸.

The most preferred compounds are: 7-(3-Methoxy-benzyl)-2,3,4,5-tetrahydro-1H-benzo[d]azepine fumarate (Compound 1) and 8-(4-Hydroxy-benzyl)-2,3,4,5-tetrahydro-1H-benzo[d]azepin-7-ol hydrobromide (Compound 5), both of which are novel.

Preferred novel compounds of Formula A are those in which R═C₁ alkyl.

### Synthesis

The compounds of this invention can be prepared by known methods 5 including those reported in WO 93/00094, WO 2005/042490, WO 2005/042491, and WO 2006/018260. The invention will be described in greater detail by way of specific examples. The following examples are offered for illustrative purposes only and are not intended to limit the invention in any manner

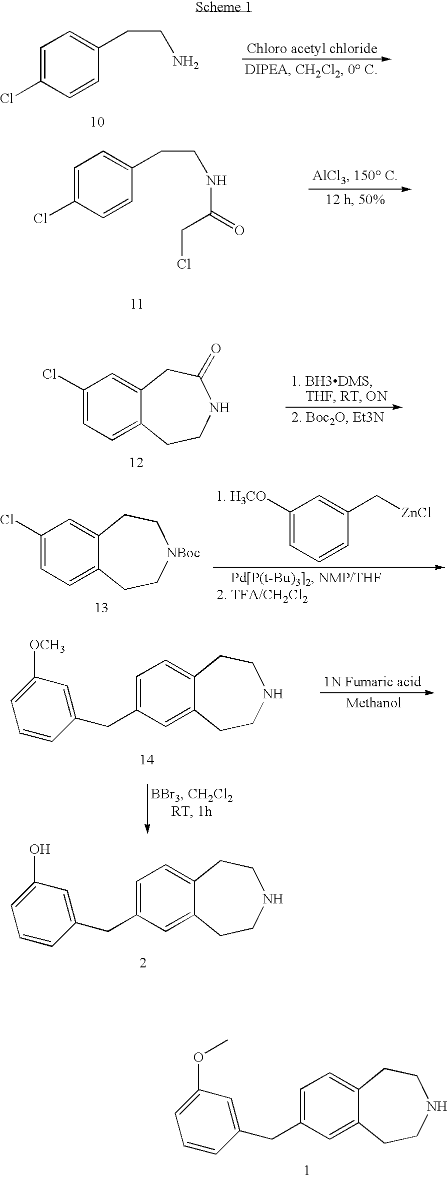

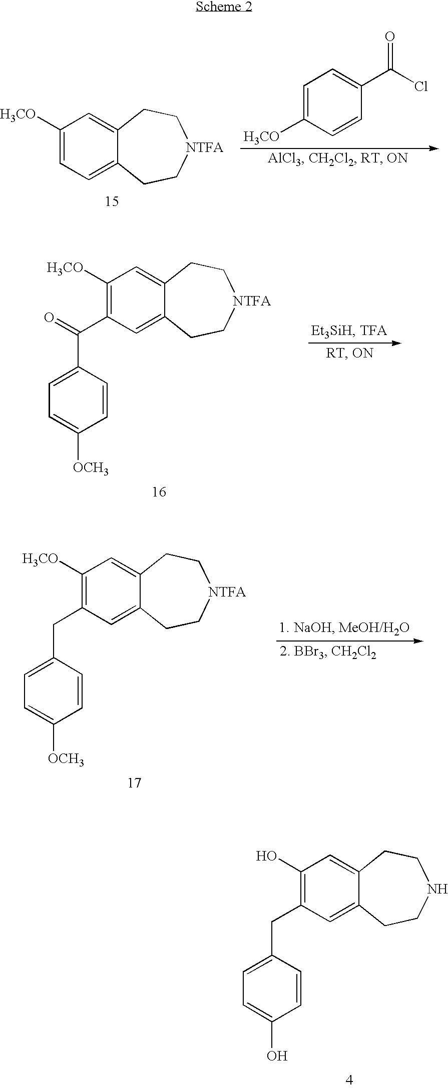

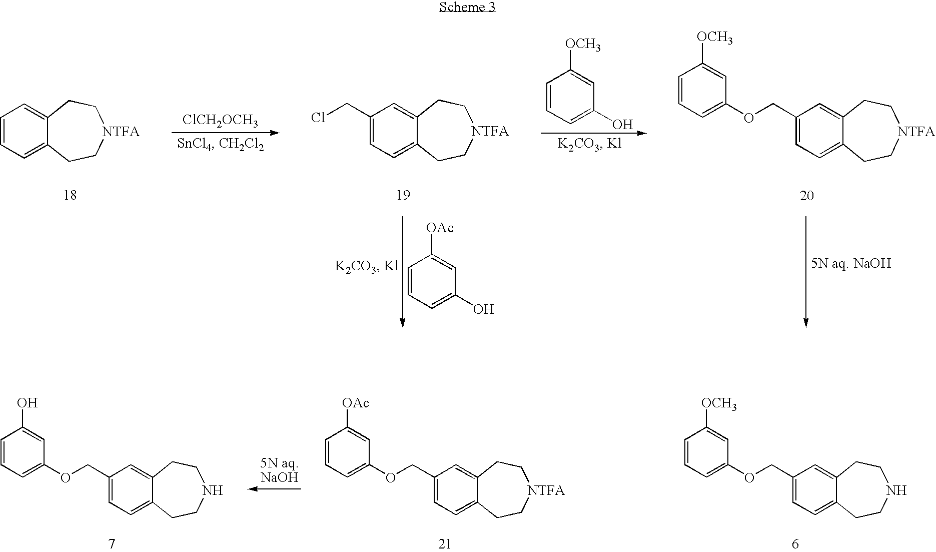

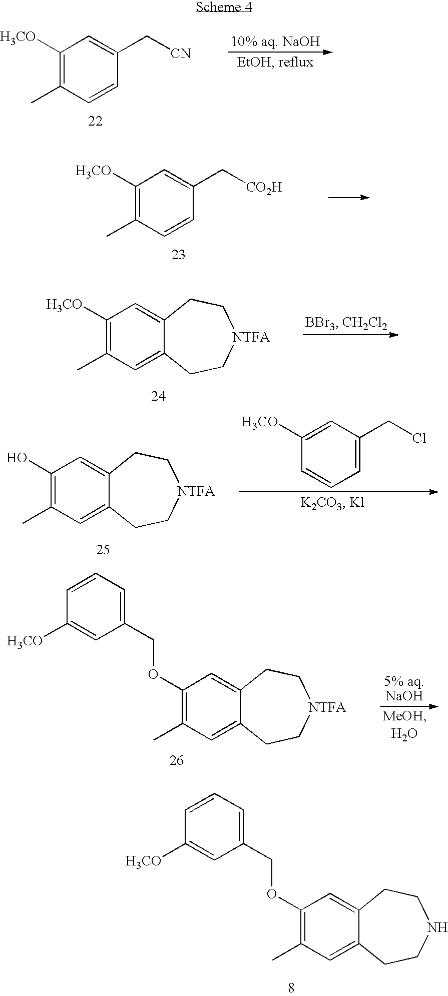

### EXAMPLE 1

### 7-(3-Methoxy-benzyl)-2,3,4,5-tetrahydro-1H-benzo[d]azepine (1)

7-(3-Methoxy-benzyl)-2,3,4,5-tetrahydro-1H-benzo[d]azepine (1) was prepared by the multiple step process outlined below.

### Preparation of 2-Chloro-N-[2-(4-chloro-phenyl)-ethyl]-acetamide (11)

To 2-(4-Chlorophenyl)-ethylamine (5 g, 32.1 mmol) in CH₃CN (150 ml) was added Et₃N (5.4 ml, 38.5 mmol) at 0° C. under a N₂ atmosphere followed by chloroacetyl chloride (2.83 ml, 35.34 mmol). The reaction mixture was stirred at 0° C. for 0.5 h and at room temperature for 3 h. Then solvent was evaporated, the crude mixture was dissolved in EtOAc (100 ml) and poured into 200 ml of H₂O. The organic layer was separated, the water layer was extracted with EtOAc, the combined organic extract was washed with H₂O, brine, dried over MgSO₄, and the solvent was removed. The crude compound was dissolved in 30 ml of EtOAc, hexane (150 ml) was added and cooled to 0° C. to precipitate the brown solid. The solid was filtered and dried to afford 4.9 g of 11 in 67% yield.

¹H NMR (CDCl₃, 400 MHz): δ2.82 (t, 2H, J=6.8 Hz), 3.54 (q, 2H, J=6.4 Hz), 4.02 (s, 2H), 6.55-6.65 (bs, 1H), 7.12-7.15 (m, 2H), 7.26-7.30 (m,2H).

### Preparation of 8-Chloro-1,3,4,5-tetrahydro-benzo[d]azepin-2-one (12)

To the acetamide 11 (4.9 g, 21.12 mmol) AlCl₃ (8.47 g, 63.36 mmol) was added and the reaction mixture was heated at 150° C. neat for 12 h. Then the reaction mixture was cooled down to room temperature and quenched by the addition of 10% aq. HCl dropwise. EtOAc (100 ml) was added to this and the layers were separated. The water layer was extracted with EtOAc (2×50 ml) and the combined organic extract was washed with water, and brine, dried over MgSO₄, and solvent was removed. The crude compound was purified by flash column chromatography using 85% EtOAc in hexane as eluent to afford 12 (3.2 g, 77.5%) as an off white solid with less than 10% other impurity.

¹H NMR (CDCl₃, 400 MHz): δ3.06-3.13 (m, 2H), 3.53-3.58 (m, 2H), 3.79 (s, 2H), 6.37 (bs, 1H), 7.03-7.17 (m, 3H). LC/MS=196 (M+1).

### Preparation of 7-Chloro-1,2,4,5-tetrahydro-benzo[d]azepine-3-carboxylic acid tert-butyl ester (13)

To the amide 12 (0.75 g, 3.84 mmol) in dry THF (50 ml) was added BH₃.DMS (13.5 ml, 13.46 mmol, 2M in toluene) at 0° C. under N₂ atmosphere and the reaction mixture was stirred at room temperature for 12 h. Then the reaction mixture was quenched by the addition of 20 ml of 10% aq. HCl and refluxed for 1 h. After the reaction mixture was brought to room temperature, the organic solvent was evaporated; the water layer was extracted with EtOAc (50 ml) to remove any unreacted starting material or non polar impurities. The water layer was basified with the addition of 1 N aq. NaOH solution and extracted with EtOAc (3×50 ml). The combined organic extract was washed with water, and brine, dried over MgSO₄, and solvent was removed. The crude oily compound (0.3 g) was dissolved in MeOH (30 ml) and Et₃N (0.58 ml, 1.37 mmol) was added followed by (Boc)₂O (0.6 g, 2.75 mmol) The reaction mixture was stirred at room temperature for 12 h. Solvents were evaporated and the crude mixture was purified by flash column chromatography using 10% EtOAc in hexane as an eluent to afford 13 (0.18 g, 40%) as a colorless gummy oil.

¹H NMR (CDCl₃, 400 MHz): δ1.48 (s, 9H), 2.85 (bs, 1.5H), 2.95 (bs, 0.5H), 3.25 (bs, 0.5H), 3.53-3.54 (m, 4H), 7.02-7.10 (m, 3H).

### Preparation of 7-(3-Methoxy-benzyl)-2,3,4,5-tetrahydro-1H-benzo[d]azepine (1)

To 3-methoxy benzylzinc chloride (J. Am. Chem. Soc., 2001, 123, 2719-2724) (1.92 ml, 0.96 mmol, 0.5M in THF) in a dry 20 ml microwave reactor vial N-methyl pyrrolidone (2.5 ml) was added and stirred for 15 minute at room temperature under N₂ atmosphere. Then to the reaction mixture Pd [P(t-Bu)₃]₂ (6.5 mg, 0.012 mmol) was added followed by the addition of 13 (0.18 g, 0.64 mmol) in 2 ml of THF. The mixture was heated to 150° C. in microwave reactor for 30 min, cooled to room temperature, and acidified by the addition of 5 ml of 10% aq. HCl. The compound was extracted with EtOAC (3×20 ml). The combined EtOAc extract was dried over MgSO₄ and the volatiles were removed under reduced pressure. The crude oil was purified by flash column chromatography and subjected to deprotection by treating with 5 eq of TFA in CH₂Cl₂ at room temperature for 4 h. Solvent was removed and the crude residue was dissolved in MeOH. Solid NaHCO₃ was added and stirred for 0.5 h to make the solution basic. The mixture was filtered, the solvent was removed and the crude residue was purified by combiflash column chromatography using 10% MeOH+5% Et₃N in EtOAc as eluent to afford the amine 14 (0.1 g) as colorless oil. The oil was taken in 1 ml of MeOH and to it 1 eq. of 1N of fumaric acid in MeOH was added. Ether (50 ml) was added to precipitate the fumarate salt which was filtered and dried to afford 1 (50 mg) as off white powder. ¹H NMR (CD₃OD, 400 MHz): δ3.09-3.13 (m, 4H), 3.26-3.28 (m, 4H), 3.76 (s, 3H), 3.91 (s, 2H), 6.70 (s, 2H), 6.74-6.79 (m, 3H). 7.08 (s, 2H), 7.14-7.18 (m, 2H). ¹³C NMR (CD₃OD, 100 MHz): δ 33.25, 33.66, 42.31, 47.55, 47.62, 55.58, 112.30, 115.80, 122.26, 128.96, 130.45, 130.76, 131.14, 137.96, 140.33, 142.19, 144.03, 161.32. LC/MS=268 (M+1). Anal. Calcd. For C₂₂H₂₅ NO₅: C, 68.91; H, 6.57; N, 3.65. Found: C, 68.48; H, 6.60; N, 3.67.

### EXAMPLE 2

### 3-(2,3,4,5-Tetrahydro-1H-benzo[d]azepin-7-ylmethyl)-phenol (2)

### Preparation of 3-(2,3,4,5-Tetrahydro-1H-benzo[d]azepin-7-ylmethyl)-phenol (2)

To compound 14 (0.12 g, 0.44 mmol) in CH₂Cl₂ (10 mL) at room temperature under N₂ atmosphere was added BBr₃ (0.064 ml, 0.67 mmol) drop wise. The reaction mixture was stirred there for 2 h, and the solvent was evaporated. The solids were dissolved in H₂O, neutralized by the addition of solid NaHCO₃, and extracted with EtOAc. The combined extract was dried over MgSO₄, the solvent was evaporated and the fumarate salt (2, 0.14 g) was made as described for 1. ¹H NMR (CD₃OD, 400 MHz): δ2.99-3.07 (m, 4H), 3.14-3.17 (m, 4H), 3.73 (s, 2H), 6.46-6.49 (m, 2H), 6.49-6.50 (m, 1H), 6.63 (s, 2H), 6.92-6.95 (m, 3H), 7.0-7.06 (m, 1H), 7.06-7.09 (m, 1H). LC/MS=254 (M+1).

### EXAMPLE 3

### 7-(3,5-Dimethoxy-benzyl)-2,3,4,5-tetrahydro-1H-benzo[d]azepine (3)

### Preparation of 7-(3,5-Dimethoxy-benzyl)-2,3,4,5-tetrahydro-1H-benzo[d]azepine (3)

Compound 3 was prepared from 13 using 3,5-dimethoxy benzyl zinc chloride following the same procedure as described for example 1.

¹H NMR (CD₃OD, 400 MHz): δ2.99-3.00 (m, 4H), 3.14-3.17 (m, 4H), 3.62 (s, 6H), 3.74 (s, 2H), 6.23-6.24 (m, 3H), 6.58 (s, 2H), 6.95-6.98 (m, 2H), 7.14-7.18 (m, 1H). ¹³C NMR (CD₃OD, 100 MHz): δ33.26, 33.66, 42.49, 47.54, 47.61, 55.66, 98.78, 108.12, 128.95, 130.73, 131.12, 137.99, 140.33, 142.04, 144.73, 162.43. LC/MS=298 (M+1). Anal. Calcd. For C₂₂H₂₅ NO₅: C, 66.81; H, 6.58; N, 3.39. Found: C, 66.09; H, 6.12; N, 3.12.

### EXAMPLE 4

### 8-(4-hydroxybenzyl)-2,3,4,5-tetrahydro-1H-3-benzazepin-7-ol (4)

8-(4-hydroxybenzyl)-2,3,4,5-tetrahydro-1H-3-benzazepin-7-ol was prepared by the multiple step procedure outlined below.

### Preparation of (4-methoxyphenyl)[8-methoxy-3-(trifluoroacetyl)-2,3,4,5-tetrahydro-1H-3-benzazepin-7-yl]methanone (16)

To compound 15 (DE 3418270, 1985) (0.38 g, 1.30 mmol) in CH₂Cl₂ (20 mL) at room temperature under N₂ atmosphere was added AlCl₃ (0.55 g, 4.15 mmol) followed by the addition of 4-methoxy benzoyl chloride (0.48 mL, 3.47 mmol). The reaction mixture was stirred at room temperature overnight, quenched with the addition of water (10 mL), and extracted with EtOAc (3×50 ml). The combined organic extract was washed with water, and brine, dried over MgSO₄ and evaporated to a crude residue. The crude mixture was purified by column chromatography using 30% EtOAc in hexane as eluent to afford the title compound 16 as an oil (0.35 g, 61.7%).

¹H NMR (CDCl₃, 400 MHz): δ2.94-2.96 (m, 2H), 3.00-3.02 (m, 2H), 3.68-3.88 (m, 4H), 3.72 (s, 3H), 3.87 (s, 3H), 6.76 (d, 1H, J=12 Hz), 6.90-6.95 (m, 2H), 7.12 (d, 1H, J=8.4 Hz), 7.78-7.80 (m, 2H). LC/MS=408 (M+1).

### Preparation of 7-methoxy-8-(4-methoxybenzyl)-3-(trifluoroacetyl)-2,3,4,5-tetrahydro-1H-3-benzazepine (17)

To the ketone 16 (0.35 g, 0.85 mmol) in trifluroacetic acid (10 mL) was added Et₃SiH (0.69 mL, 4.29 mmol) and the reaction mixture was stirred at room temperature for 12 h. The solvent was evaporated and the crude mixture was purified by combiflash chromatography to afford 0.26 g of 17 as light yellow oil.

¹H NMR (CDCl₃, 400 MHz): δ2.82-2.84 (m, 2H), 2.90-2.94 (m, 2H), 3.61-3.72 (m, 4H), 3.77 (s, 3H), 3.81 (s, 3H), 3.85 (s, 2H), 6.64 (d, 1H, J=12.8 Hz), 6.78-6.82 (m, 3H), 7.10-7.13 (m, 2H). LC/MS=411 (M+18).

### Preparation of 8-(4-hydroxybenzyl)-2,3,4,5-tetrahydro-1H-3-benzazepin-7-ol (4)

To compound 17 (0.1 g, 0.25 mmol) in methanol:water (4:1, 10 mL) was added 5 ml of 5N aq. NaOH solution and the reaction mixture was stirred at room temperature for 12 h. Then methanol was removed and compound was extracted with EtOAc (3×15 mL). The combined extract was washed with water, brine, dried over MgSO₄ and evaporated to afford the crude amine. The crude amine was dissolved in 15 mL of CH₂Cl₂, BBr₃ (0.085 mL, 0.9 mmol) was added and the resulting solution was stirred at room temperature for 2 h. The reaction mixture was quenched with 2 mL of methanol, and the volatiles were removed. The crude solid was dissolved in 1 ml of methanol and ether (20 mL) was added to precipitate the HBr salt. Solids were filtered, washed with ether and dried in vacuum to afford 5 (34 mg) as off white powder.

¹H NMR (CD₃OD, 400 MHz): δ2.99-3.06 (m, 4H), 3.18-3.25 (m, 4H), 3.82 (s, 2H), 6.63 (s, 1H), 6.72-6.79 (m, 3H), 7.04-7.06 (m, 2H), 7.45 (s, 1H). ¹³C NMR (CD₃OD, 100 MHz): δ 32.22, 32.67, 34.69, 47.03, 47.37, 115.61, 116.83, 127.94, 129.73, 130.36, 132.09, 132.44, 137.70, 154.36, 155.39. LC/MS=270 (M+1). Anal. Calcd. For C₁₇H₂₀ NO₂, 0.33 mol of H₂O: C, 57.31; H, 5.85; N, 3.93. Found: C, 57.23; H, 5.84; N, 3.79.

### EXAMPLE 5

### 8-(3-hydroxybenzyl)-2,3,4,5-tetrahydro-1H-3-benzazepin-7-ol (5)

### Preparation of 8-(3-hydroxybenzyl)-2,3,4,5-tetrahydro-1H-3-benzazepin-7-ol (5)

Compound 5 was made from 15 following the same procedures described for 4 using 3-methoxy benzoyl chloride as the acylating reagent.

¹H NMR (CD₃OD, 400 MHz): δ2.99-3.06 (m, 4H), 3.21-3.23 (m, 4H), 3.72 (s, 2H), 6.55-6.57 (m, 1H), 6.59-6.63 (m, 3H), 6.76 (s, 1H), 6.93-6.95 (m, 1H). ¹³C NMR (CD₃OD, 100 MHz): δ 32.81, 33.37, 35.97, 47.73, 48.07, 113.66, 116.80, 117.31, 121.23, 127.98, 130.13, 130.76, 132.91, 138.94, 144.18, 155.31, 158.33. LC/MS=270 (M+1). Anal. Calcd. For C₁₇H₂₀ NO₂, 0.33 mol of H₂O: C, 57.31; H, 5.85; N, 3.93. Found: C, 57.16; H, 5.81; N, 3.74.

### EXAMPLE 6

### 3-(trifluoroacetyl)-2,3,4,5-tetrahydro-1H-3-benzazepine (18)

The preparation of 3-(trifluoroacetyl)-2,3,4,5-tetrahydro-1H-3-benzazepine (18) was by the multiple step process described below.

Compound 18 was prepared from phenyl ethylamine following the same procedure as described for 15.

¹H NMR (CDCl₃, 400 MHz): δ2.96-3.0 (m, 4H), 3.68-3.70 (m, 2H), 3.76-3.79 (m, 2H), 7.12-7.19 (m, 4H).

### Preparation of 7-(chloromethyl)-3-(trifluoroacetyl)-2,3,4,5-tetrahydro-1H-3-benzazepine (19)

To the compound 18 (0.5 g, 2.05 mmol) in CH₂Cl₂ (20 mL) at −10° C. was added SnCl₄ (0.84 mL, 7.20 mmol) followed by ClCH₂OCH₃ (0.24 mL, 5.14 mmol). The reaction mixture was stirred at room temperature for 24 h, quenched with water, and extracted with EtOAc (3×25 ml). The combined extract was washed with water, and brine, dried over MgSO₄. The volatiles were evaporated to afford residue which was purified by flash column chromatography using 10% EtOAc in hexane as eluent to afford 19 (0.5 g) as an oil.

¹H NMR (CDCl₃, 400 MHz): δ2.95-3.02 (m, 4H), 3.69-3.70 (m, 2H), 3.75-3.79 (m, 2H), 4.55 (s, 2H), 7.12-7.22 (m, 3H).

### Preparation of 7-[(3-methoxyphenoxy)methyl]-3-(trifluoroacetyl)-2,3,4,5-tetrahydro-1H-3-benzazepine (20)

To the compound 19 (0.2 g, 0.68 mmol) in CH₃CN (20 mL) was added K₂CO₃ (0.28 g, 2.06 mmol) followed by 3-methoxy phenol (0.09 mL, 0.82 mmol) and KI (0.12 g, 0.75 mmol) and the reaction mixture was stirred at room temperature for 12 h. Solids were filtered and solvents were evaporated to a crude mixture which was purified by flash column chromatography using 15% EtOAc in hexane as eluent to afford 20 (0.18 g).

¹H NMR (CDCl₃, 400 MHz): δ2.96-3.01 (m, 4H), 3.69-3.70 (m, 2H), 3.75-3.79 (m, 5H), 4.99 (s, 2H), 6.56-6.58 (m, 3H), 7.12-7.25 (m, 4H). LC/MS=380 (M+1).

### Preparation of 7-[(3-methoxyphenoxy)methyl]-2,3,4,5-tetrahydro-1H-3-benzazepine (6)

To the compound 20 (0.18 g, 0.47 mmol) in methanol:water (4:1, 10 mL) was added 5 ml of 5N aq. NaOH solution and the reaction mixture was stirred at room temperature for 12 h. Then methanol was removed and compound was extracted with EtOAc (3×15 ml). The combined extract was washed with water, and brine, dried over MgSO₄ and evaporated to yield the crude amine. The amine was purified by column chromatography using a mixture of 10% methanol, 5% Et₃N and 85% EtOAc as eluent to afford the amine 6. The amine was converted to its fumarate salt (77 mg) as described for compound 1.

¹H NMR (CD₃OD, 400 MHz): δ3.16-3.19 (m, 4H), 3.30-3.32 (m, 4H), 3.77 (s, 3H), 5.05 (s, 2H), 6.51-6.54 (m, 3H), 6.70 (s, 2H), 7.14-7.18 (m, 1H), 7.24-7.26 (m, 1H), 7.30-7.33 (m, 2H). ¹³C NMR (CD₃OD, 100 MHz): δ33.37, 33.66, 47.50, 55.69, 70.45, 102.40, 107.45, 108.18, 127.70, 129.80, 130.82, 130.94, 137.50, 139.90, 141.12, 161.56, 163.32. LC/MS=284 (M+1). Anal. Calcd. For C₂₂H₂₅ NO₆, 0.12 mol of H₂O: C, 65.78; H, 6.34; N, 3.49. Found: C, 65.87; H, 6.31; N, 3.47.

### EXAMPLE 7

### 3-(2,3,4,5-tetrahydro-1H-3-benzazepin-7-ylmethoxy)phenol (7)

3-(2,3,4,5-Tetrahydro-1H-3-benzazepin-7-ylmethoxy)phenol (7) was prepared by the multiple step process outlined below.

### Preparation of 3-{[3-(trifluoroacetyl)-2,3,4,5-tetrahydro-1H-3-benzazepin-7-yl]methoxy}phenyl acetate (21)

Compound 21 was made from 19 following the same procedure described for 20 using resorcinol monoacetate as alkylating agent.

¹H NMR (CDCl₃, 400 MHz): δ2.28 (s, 3H)2.96-3.01 (m, 4H), 3.69-3.70 (m, 2H), 3.77-3.78 (m, 2H), 4.99 (s, 2H), 6.72-6.73 (m, 2H), 6.83-6.85 (m, 1H), 7.19-7.23 (m, 4H). LC/MS=408 (M+1).

### Preparation of 3-(2,3,4,5-tetrahydro-1H-3-benzazepin-7-ylmethoxy)phenol (7)

Compound 7 was prepared from 21 following the same procedure as described for compound 6.

¹H NMR (CD₃OD, 400 MHz): δ3.15-3.18 (m, 4H), 3.29-3.31 (m, 4H), 4.96 (s, 2H), 6.26-6.47 (m, 3H), 6.70 (s, 2H), 7.04-7.11 (m, 1H), 7.23-7.35 (m, 5 4H). LC/MS=270 (M+1). Anal. Calcd. For C₂₁H₂₃ NO₆, 0.33 mol of H₂O: C, 64.44; H, 6.09; N, 3.58. Found: C, 64.06; H, 6.02; N, 3.76.

### EXAMPLE 8

### 7-[(3-methoxybenzyl)oxy]-8-methyl-2,3,4,5-tetrahydro-1H-3-benzazepine (8)

7-[(3-Methoxybenzyl)oxy]-8-methyl-2,3,4,5-tetrahydro-1H-3-benzazepine (8) was prepared by the multiple step process outlined below.

### Preparation of (3-methoxy-4-methylphenyl)acetic acid (23)

To 3-methoxy-4-methylphenyl acetonitrile (3 g, 18.63 mmol) in ethanol (100 mL) was added 20 mL of 10% aqueous NaOH solution. The reaction mixture was refluxed for 20 h. Ethanol was removed, the crude mixture was dissolved in water (100 mL) and adjusted to pH 4 by adding conc. HCl. Solids that formed were filtered, washed with water, and dried in vacuum to give 23 (2.64 g) as off white solid.

¹H NMR (CDCl₃, 400 MHz): δ2.18 (s, 3H), 3.60 (s, 2H), 3.81 (s, 3H), 6.73-6.77 (m, 2H), 7.07 (d, 1H, J=7.6 Hz). LC/MS=180 (M+1).

### Preparation of 7-methoxy-8-methyl-3-(trifluoroacetyl)-2,3,4,5-tetrahydro-1H-3-benzazepine (24)

Compound 24 was prepared from 23 following the same procedures as compound 15.

¹H NMR (CDCl₃, 400 MHz): δ2.17 (s, 3H), 2.86-2.95 (m, 4H), 3.64-3.69 (m, 2H), 3.72-3.81 (m, 2H), 3.81 (s, 3H), 6.60 (d, 1H, J=12 Hz), 6.90 (d, 1H, J=13.2 Hz). LC/MS=288 (M+1).

### Preparation of 8-methyl-3-(trifluoroacetyl)-2,3,4,5-tetrahydro-1H-3-benzazepin-7-ol (25)

To compound 24 (0.5 g, 1.74 mmol) in CH₂Cl₂ (30 mL) at 0° C. was added BBr₃ (0.25 mL, 2.61 mmol). The reaction mixture was stirred at room temperature for 3 h, and quenched with 5 mL of methanol. The solvent was removed and the crude mixture was purified by chromatography to afford 25 (0.45 g).

¹H NMR (CDCl₃, 400 MHz): δ2.20 (s, 3H), 2.85-2.89 (m, 4H), 3.63-3.67 (m, 2H), 3.71-3.75 (m, 2H), 3.69-4.71 (m, 1H), 6.59 (d, 1H, J=7.6 Hz), 6.88 (d, 1H, J=12.4 Hz). LC/MS=274 (M+1).

### Preparation of 7-[(3-methoxybenzyl)oxy]-8-methyl-3-(trifluoroacetyl)-2,3,4,5-tetrahydro-1H-3-benzazepine (26)

Compound 26 was made from 25 using 3-methoxy benzyl bromide as alkylating agent following the same procedure as described for compound 20.

¹H NMR (CDCl₃, 400 MHz): δ2.24 (s, 3H), 2.86-2.92 (m, 4H), 3.64-3.67 (m, 2H), 3.72-3.76 (m, 2H), 3.82 (s, 3H), 5.02 (s, 2H), 6.94 (d, 1H, J=12 Hz), 7.0-7.02 (m, 2H), 7.28-7.31 (m, 1H). LC/MS=394 (M+1).

### Preparation of 7-[(3-methoxybenzyl)oxy]-8-methyl-2,3,4,5-tetrahydro-1H-3-benzazepine (8)

Compound 8 was prepared from 26 following the same procedure as described for compound 6.

¹H NMR (CD₃OD, 400 MHz): δ2.94-2.99 (m, 4H), 3.13-3.17 (m, 4H), 3.71 (s, 3H), 4.98 (s, 2H), 6.58 (s, 1.5H), 6.73 (s, 1H), 6.78-6.82 (m, 1H), 6.91-6.92 (m, 3H), 7.17-7.19 (m, 1H). ¹³C NMR (CD₃OD, 100 MHz): δ15.95, 32.81, 33.63, 47.63, 47.85, 55.67, 70.96, 113.81, 114.11, 114.31, 120.39, 126.68, 130.59, 132.15, 133.11, 138.72, 140.51, 157.17, 161.36. LC/MS=298 (M+1). Anal. Calcd. For C₁₉H₂₃ NO₂, 0.85 mol of fumaric acid: C, 67.87; H, 6.71; N, 3.53. Found: C, 67.81; H, 6.74; N, 3.63.

The compounds of this invention can be incorporated into various types of ophthalmic formulations for delivery to the eye (e.g., topically, intracamerally, or via an implant). The compounds are preferably incorporated into topical ophthalmic formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a benzodifuran analog in a physiologically acceptable isotonic aqueous buffer. Further, the ophthalmic solution may include an ophthalmologically acceptable surfactant to assist in dissolving the benzodifuran analog. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the compound of Formula A in a hydrophilic base prepared from the combination of, for example, carbopol-974, or the like, according to the published formulations for analogous ophthalmic preparations; preservatives and tonicity agents can be incorporated.

The compounds of the present invention are preferably formulated as topical ophthalmic suspensions or solutions, with a pH of about 4 to 8. The compounds will normally be contained in these formulations in an amount 0.01 to 5% (w/v), but preferably in an amount of 0.1 to 2% (w/v). Thus, for topical presentation 1 to 2 drops of these formulations would be delivered to the surface of the eye 1 to 4 times per day according to the discretion of a skilled clinician.

The compounds of Formula A can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), cc1 antagonists (e.g. nipradolol), α₂ agonists (e.g., iopidine and brimonidine), miotics (e.g., pilocarpine and epinephrine), prostaglandin analogs (e.g., latanoprost, travoprost, unoprostone, and compounds set forth in U.S. Pat. Nos. 5,889,052; 5,296,504; 5,422,368; and 5,151,444, “hypotensive lipids” (e.g., lumigan and compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203350, and is appropriate compounds from WO94/13275, including memantine.

The following methods can be used to characterize the compounds of the present invention.

### Method 1

### r5-HT_{2A} Functional Assay: FLIPR

### Rat Vascular Smooth Muscle Cells

The receptor-mediated mobilization of intracellular calcium ([Ca²⁺]ᵢ) was studied using the Fluorescence Imaging Plate Reader (FLIPR) instrument. Rat vascular smooth muscle cells, A7r5, were grown in a normal media of DMEM/10% FBS and 10 μg/ml gentamycin. Confluent cell monolayers were trypsinized, pelleted, and re-suspended in normal media. Cells were seeded in a 50 μL volume at a density of 20,000 cells per well in a black wall, 96-well tissue culture plate and grown for 2 days. On the day of the experiment, one vial of FLIPR Calcium Assay Kit dye was re-suspended in 50 ml of a FLIPR buffer consisting of Hank's Balanced Salt Solution (HBSS), 20 mM HEPES, and 2.5 mM probenecid, pH 7.4. Cells were loaded with the calcium-sensitive dye by addition of an equal volume (50 μl) to each well of the 96-well plate and incubated with dye for 1 h at 23° C. Typically, test compounds were stored at 25 μM in 50% DMSO/50% Ethanol solvent. Compounds were diluted 1:50 in 20% DMSO/20% Ethanol. For dose-response experiments, compounds were diluted 1:50 in FLIPR buffer and serially diluted 1:10 to give a 5- or 8-point dose-response curve.

At the beginning of an experimental run, a signal test was performed to check the basal fluorescence signal from the dye-loaded cells and the uniformity of the signal across the plate. The basal fluorescence was adjusted between 8000-12000 counts by modifying the exposure time, the camera F-stop, or the laser power. The instrument settings for a typical assay were as follows: laser power 0.3-0.6 W, camera F-stop F/2, and exposure time 0.4 sec. An aliquot (25 μl) of the test compound was added to the existing 100 μl dye-loaded cells at a dispensing speed of 50 μl/sec. Fluorescence data were collected in real-time at 1.0 sec intervals for the first 60 sec and at 6.0 sec intervals for an additional 120 sec. Responses were measured as peak fluorescence intensity minus basal and where appropriate were expressed as a percentage of a maximum 5-HT-induced response.

### Method 2

### r5-HT_{2C} Functional Assay: FLIPR

This assay were performed as for the r5-HT_{2A} receptor above, except that SR3T3 cells expressing the recombinant rat 5-HT_{2C} receptor were utilized.

### Method 2

### h5-HT₂ Functional Assay

Functional response at the 5-HT₂ receptor subtypes was determined using CHO-K1 cells stably expressing mitochondrially-targeted bioluminescent aequorin, G_{α16}, and one of either human serotonin receptor clone 5-HT_{2A}, 5-HT_{2B} , or 5-HT_{2C}. Prior to testing, cells were loaded in suspension with coelenterazine for 4-16 hours and directly injected onto different concentrations of the test compound. Light emitted from the cells was measured 20-30 seconds following receptor activation. A luminometer (Hamamatsu, FDSS-6000) was used to record luminescence in response to the test compound. The mean response signal at each of 8-11 different concentrations was integrated to provide an estimation of receptor activation, expressed as the EC₅₀ value. The efficacy of the response (Eₘₐₓ) at the 5-HT_{2A} and 5-HT_{2B} receptors is expressed relative to the response of α-methyl-5-HT under the same assay conditions while the efficacy at 5-HT_{2C} is expressed relative to the response of 5-HT.

The above procedures were used to generate the data shown in Table 1.

TABLE 1
5-HT2 Functional Data.

| | | r5-HT | | h5-HT | | |
|---|---|---|---|---|---|---|
| | | 2A | 2C | 2A | 2B | 2C |
| | | EC₅₀ nm | EC₅₀ nm | EC₅₀ nm | EC₅₀ nm | EC₅₀ nm |
| Example | Structure | (Eₘₐₓ) | (Eₘₐₓ) | (Eₘₐₓ) | (Eₘₐₓ) | (Eₘₐₓ) |
| 1 | 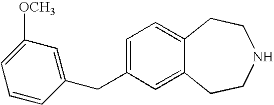 | 386(39) | 82(100) | 1.5(102) | 390(87) | 1.1(105) |
| 2 | 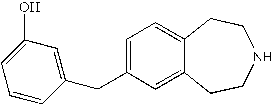 | >1000 | 120(100) | 31(98) | 1242(35) | 8.8(109) |
| 3 | 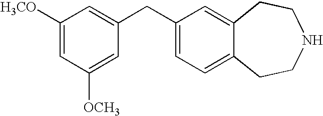 | >10,000 | 629(70) | 16.9(104) | >10000(6) | 20(105) |
| 4 | 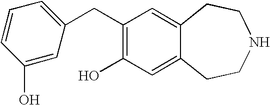 | 1.13(45%) | 1.48(81) | 1.2(106) | 43(73) | 0.3(110) |
| 5 | 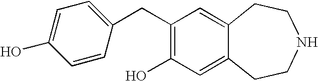 | 8.9(45) | 0.8(108) | 1.2(106) | 8.4(84) | 0.22(108) |
| 6 | 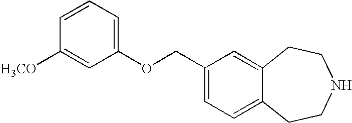 | 69(35%) | 91(89%) | 0.4(105) | 417(61) | 3.9(107) |
| 7 | 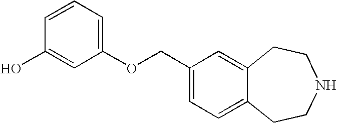 | 440(40%) | 65(92) | — | — | — |
| 8 | 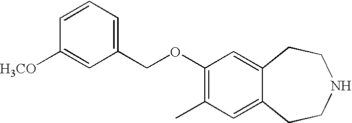 | 1920(48%) | 250(102) | 2.4(102) | 230(63) | 4.5(101) |

### Method 3

### Acute IOP Response in Lasered (Hypertensive) Eyes of Conscious Cynomolgus Monkeys

Intraocular pressure (IOP) can be determined with an Alcon Pneumatonometer after light corneal anesthesia with 0.1% proparacaine. Eyes are washed with saline after each measurement. After a baseline IOP measurement, test compound is instilled in one 30 μL aliquot to the right eyes only of nine cynomolgus monkeys. Vehicle is instilled in the right eyes of six is additional animals. Subsequent IOP measurements are taken at 1, 3, and 6 hours.

The above method was used to determine the IOP lowering efficacy of Compound 1. All eyes were pretreated with 1 drop of 0.5% proparacaine to address discomfort. The results are shown in Table 2.

TABLE 2
IOP Efficacy.

| | | Baseline IOP | % IOP change (mmHg) | | |
|---|---|---|---|---|---|
| Compound | Dose ( μg) | (mmHg) | 1 hr | 3 hr | 6 hr |
| 1 | 100 | 38.3 | −16.5 | −33.1 | −42.5 |
| | | | (6.6) | (13.1) | (16.8) |
| 1 | 150 | 40.4 | −15.6 | −29.5 | −37.8 |
| | | | (6.1) | (12.3) | (15.6) |

The following topical ophthalmic formulations are useful according to the present invention administered 1-4 times per day according to the discretion of a skilled clinician.

### EXAMPLE 9

| Ingredients | Amount (wt %) |
|---|---|
| Compound 1 | 0.1–2 |
| Hydroxypropyl methylcellulose | 0.5 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8–7.4 |
| Purified water | q.s. to 100 |

### EXAMPLE 10

| Ingredients | Amount (wt %) |
|---|---|
| Compound 1 | 0.1–2 |
| Methyl cellulose | 4.0 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8–7.4 |
| Purified water | q.s. to 100 |

### EXAMPLE 11

| Ingredients | Amount (wt %) |
|---|---|
| Compound 1 | 0.1–2 |
| Guar gum | 0.4–6.0 |
| Dibasic sodium phosphate | 0.2 |
| (anhydrous) | |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8–7.4 |
| Purified water | q.s. to 100 |

### EXAMPLE 12

| Ingredients | Amount (wt %) |
|---|---|
| Compound 1 | 0.1–2 |
| White petrolatum and mineral oil and | Ointment consistency |
| lanolin | |
| Dibasic sodium phosphate (anhydrous) | 0.2 |
| Sodium chloride | 0.5 |
| Disodium EDTA (Edetate disodium) | 0.01 |
| Polysorbate 80 | 0.05 |
| Benzalkonium chloride | 0.01 |
| Sodium hydroxide/Hydrochloric acid | For adjusting pH to 6.8–7.4 |

We claim:

1. A method for lowering or controlling intraocular pressure in a warm-blooded mammal's eye, which comprises administering to the mammal a composition comprising a pharmaceutically acceptable carrier and a pharmaceutically effective amount of a compound of formula A: 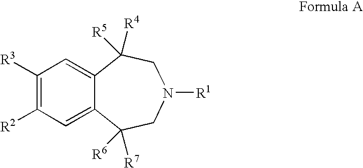
wherein:
R¹═H or C₁₋₄ alkyl;
R²═H, OH, or OR where R═C₁₋₄ alkyl;
R³═—X—Ar, —OR⁸, —(CH₂)ₙOR⁸, or —(CH₂)_{n′}—O—(CH₂)ₘOR⁸;
R⁴, R⁵═H or C₁₋₂ alkyl;
R⁶, R⁷═H or C₁₋₂ alkyl;
when R⁴ or R⁵═C₁₋₂ alkyl, then R⁶═R⁷═H;
when R⁶ or R⁷═C₁₋₂ alkyl, then R⁴═R⁵═H;
R⁸, R⁹, R¹⁰═H or C₁₋₄ alkyl;
n=1-4;
n′=1-4;
m=1-4;
X═O, —C(R⁹)(R¹⁰)—, —OC(R⁹)(R¹⁰)—, or —C(R⁹)(R¹⁰)O—;
Ar=phenyl, optionally mono- or di-substituted with F, Cl, Br, I, C₁₋₄ alkyl, OH, or OR⁸; or 2-, 3-, 4-pyridyl, optionally mono- or di-substituted with F, Cl, Br, I, C₁₋₄ alkyl, OH, or OR⁸;
and pharmaceutically acceptable salts thereof.

2. The method of claim 1 wherein for the compound of formula A R₁═H;
R₂═OH, or OR, where R═C₁₋₄ alkyl;
R⁴, R⁵═H or C₁ alkyl;
R⁶, R⁷═H or C₁ alkyl;
when R⁴ or R⁵═C₁ alkyl, then R⁶═R⁷═H;
when R⁶ or R⁷═C₁ alkyl, then R⁴═R⁵═H;
R⁸═C₁₋₄ alkyl; and
Ar=phenyl, optionally mono- or di-substituted with F, Cl, Br, C₁₋₂ alkyl, OH, or OR⁸.

3. The method of claim 1 wherein the compound is selected from the group consisting of: 7-(3-Methoxy-benzyl)-2,3,4,5-tetrahydro-1H-benzo[d]azepine fumarate; 8-(4-Hydroxy-benzyl)-2,3,4,5-tetrahydro-1H-benzo[d]azepin-7-ol hydrobromide; and pharmaceutically acceptable salts thereof.

4. The method of claim 1 wherein the mammal is a human, the is composition is a topically administered ophthalmic composition, and the pharmaceutically effective amount of the compound is 0.01-5% (w/v).

5. The method of claim 4 wherein the pharmaceutically effective amount of the compound is 0.1-2% (w/v).

6. A method for treating glaucoma in a mammal, which comprises administering to the mammal a composition comprising a pharmaceutically acceptable carrier and a pharmaceutically effective amount of a compound of formula A: 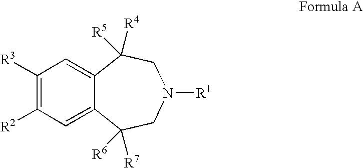
wherein
R¹═H or C₁₋₄ alkyl;
R²═H, OH, or OR where R═C₁₋₄ alkyl;
R³═—X—Ar, —OR⁸, —(CH₂)ₙOR⁸, or —(CH₂)_{n′}—O—(CH₂)ₘOR⁸;
R⁴, R⁵═H or C₁₋₂ alkyl;
R⁶, R⁷═H or C₁₋₂ alkyl;
when R⁴ or R⁵═C₁₋₂ alkyl then R⁶═R⁷═H;
when R⁶ or R⁷═C₁₋₂ alkyl, then R⁴═R⁵═H;
R⁸, R⁹, R¹⁰═H or C₁₋₄ alkyl;
n=1-4;
n′=1-4;
m=1-4;
X═O, —C(R⁹)(R¹⁰)—, —OC(R⁹)(R¹⁰)—, or —C(R⁹)(R¹⁰)O—;
Ar=phenyl, optionally mono- or di-substituted with F, Cl, Br, I, C₁₋₄ alkyl, OH, or OR⁸; or 2-, 3-, 4-pyridyl, optionally mono- or di-substituted with F, Cl, Br, I, C₁₋₄alkyl, OH, or OR⁸;
and pharmaceutically acceptable salts thereof.

7. The method of claim 6 wherein for the compound of formula A R₁═H;
R₂═OH, or OR, where R═C₁₋₄ alkyl;
R⁴, R⁵═H or C₁ alkyl;
R⁶, R⁷═H or C₁ alkyl;
when R⁴ or R⁵═C₁ alky, then R⁶═R⁷═H;
when R⁶ or R⁷═C₁ alkyl, then R⁴═R⁵═H;
R⁸═C₁₋₄ alkyl; and
Ar=phenyl, optionally mono- or di-substituted with F, Cl, Br, C₁₂ alkyl, OH, or OR⁸.

8. The method of claim 6 wherein the compound is selected from the group consisting of: 7-(3-Methoxy-benzyl)-2,3,4,5-tetrahydro-1H-benzo[d]azepine fumarate; 8-(4-Hydroxy-benzyl)-2,3,4,5-tetrahydro-1H-benzo[d]azepin-7-ol hydrobromide; and pharmaceutically acceptable salts thereof.

9. The method of claim 6 wherein the mammal is a human, the composition is a topically administered ophthalmic composition, and the pharmaceutically effective amount of the compound is 0.01-5% (w/v).

10. The method of claim 9 wherein the pharmaceutically effective amount of the compound is 0.1-2% (w/v).

11. A compound of the formula: 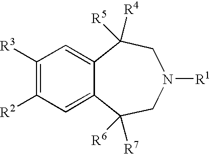
wherein
R¹═H;
R²═H, OH, or OR where R═C₁₋₄ alkyl;
R³═—X—Ar, —OR⁸, —(CH₂)ₙOR⁸, or —(CH₂)_{n′}—O—(CH₂)ₘOR⁸;
R⁴, R⁵═H;
R⁶, R⁷═H;
R⁸═C₁₋₄ alkyl;
R⁹, R¹⁰═H or C₁₋₄ alkyl;
n=1-4;
n′=1-4;
m=2-4;
X═O, —C(R⁹)(R¹⁰)—, —OC(R⁹)(R¹⁰)—, or —C(R⁹)(R¹⁰)O—;
Ar=phenyl, optionally mono- or di-substituted with F, Cl, Br, C₁₋₂ alkyl, OH, or OR⁸; or 2-, 3-, 4-pyridyl, optionally mono- or di-substituted with F, Cl, Br, I, C₁₋₄ alkyl, OH, or OR⁸;
and pharmaceutically acceptable salts thereof.

12. The compound of claim 11 wherein R═C₁ alkyl.

13. The compound of claim 11 selected from the group consisting of: 7-(3-Methoxy-benzyl)-2,3,4,5-tetrahydro-1H-benzo[d]azepine fumarate; 8-(4-Hydroxy-benzyl)-2,3,4,5-tetrahydro-1H-benzo[d]azepin-7-ol hydrobromide; and pharmaceutically acceptable salts thereof.
